(19) United States
(12) Patent Application Publication
(10) Pub. No.: US 2004/0267214 A1
(43) Pub. Date: Dec. 30, 2004

(54) Ophthalmic device for administering fluid eye medication to the human eye

(76) Inventor: Geert Kerssies, Vianen (NL)

Correspondence Address: KALOW & SPRINGUT LLP, 488 MADISON AVENUE, 19TH FLOOR, NEW YORK, NY, 10022, US

(21) Appl. No.: 10/497,289

(22) PCT Filed: Dec. 4, 2002
(86) PCT No.: PCT/NL02/00789

(30) Foreign Application Priority Data
Dec. 4, 2001 (NL) 101948.3
Mar. 11, 2002 (NL) 1020146

(51) Int. Cl. A61M 35/00

(52) U.S. Cl. 604/299; 604/295

(57) ABSTRACT

An ophthalmic device is provided for administering fluid eye medication from a dispenser (5) to a human eye, said device comprising a housing (1) comprising a base portion (2) having a first opening (4), whereby the dispenser (5) is releasably engaged with the base portion (2) and the nozzle of the dispenser is assembled through the said first opening (4), and at least one positioning member (3) extending outwardly from the base portion (5) having an edge at the outer end to be placed against the head of a user adjacent to the eye to keep the said dispenser (5) at a predetermined distance from the eye, wherein said base portion (2) further comprises a second opening (8) providing a target for directing the eye upon administering the fluid eye medication to the eye, and in that said base portion (2) has, on its side directed towards the eye, an entirely or partially reflecting and/or colour-discriminating surface.

The invention relates to an ophthalmic device for administering fluid eye medication from a dispenser to a human eye, said device comprising a housing comprising a base portion having a first opening, whereby the dispenser is releasably engaged with the base portion and the nozzle of the dispenser is assembled through the said first opening, and at least one positioning member extending outwardly from the base portion having an edge at the outer end to be placed against the head of a user adjacent to the eye to keep the said dispenser at a predetermined distance from the eye.

There are many known variants of devices such as this. For instance, U.S. Pat. No. 5,713,495 discloses the structure of an eye-drop dispenser guide which comprises among other features a partially cylindrical positioning member, which extends from the guide in a direction that is in fact an extension to the sides of the dispenser, whereby the positioning member is placed under the eye in order to keep the lower eyelid open when inserting eye-drops into the eye.

U.S. Pat. No. 5,848,999 discloses the structure of a device, which can be attached to an eye-drop squeeze bottle. This device comprises a cylindrical, dome-shaped collar instead of the cap of the dispenser and which includes an opening adapted to receive a dispenser nozzle of the dispenser. The collar further comprises a positioning member whose outer end is provided with a roughly triangular, contoured pad, facilitating the device to be held under the lower eyelid against the head, thus maintaining the squeeze bottle at a semi-fixed distance from the eye and allowing easy administration of the eye-drops.

Dutch Patent Application No. 7804368 discloses the structure of a device where a positioning member is attached to an eye-drop-dispensing container and the positioning member includes flexible pivoting extension devices with outer end pressure pads, which can be placed against the outer surface of the patient's eyelids.

Dutch Patent Applications Nos. 6512222 and 6710911 disclose the structure of such devices wherein the open end of a cone-shaped diffuser can be placed onto the orbita, allowing the eyelids to be held open.

U.S. Pat. No. 6,033,389 discloses the use of a similar, in whole or in part closed, basin-shaped diffuser, with an oval-shaped open end that can be fitted over the eye. The base of the cup has an opening through which the discharge orifice of a fluid eye medication dispenser can be assembled and sealed.

U.S. Pat. No. 5,902,292 discloses the use of a universal and easy-to-use aid that allows the hand of the user to be stabilised whilst inserting the eye-drops into the eye. The aid comprises three perpendicular step-shaped sections, one end includes an opening through which the end of an eye-drop dispenser can be placed, with the other end resting on the bridge of the user's nose while one intermediate part is designed to determine the distance to the eye.

U.S. Pat. No. 4,531,944 and EP-A-0 347 084 disclose ophthalmic devices including a housing having top, side walls and an open bottom, wherein the lower periphery of the side walls being contoured to conform to the facial area surrounding the eye socket. In the former reference the housing top includes an aperture for receiving the dispensing end of an eye drop dispenser and a second hole forming part of a vertical alignment indicator means. The latter reference the housing top comprises a first port providing an inlet for a dispensing unit for material to be supplied to the eye, and a second port being of a substantially smaller diameter serving as a target for directing the eye during the application of the material to the eye.

Various references disclose ophthalmic devices in which the centre line of the eye drop dispenser is not perpendicular to the bottom plane of the housing, which otherwise necessitate a substantial vertical position of the dispenser upon releasing droplets to the eye of a user with concommittant inconvenient lifting of an arm above the head, but has a certain angle to the perpendicular line. See, e.g. BE 1012316; U.S. Pat. Nos. 4,052,985; 5,611,788; 5,836,927; FR 1.259.476.

Pharmaceutisch Weekblad No. 28, 1016-1020 (2000) and No. 30, 1096-1099 (2000) discuss the problems that glaucoma patients experience when applying eye-drops from a dispenser in the shape of a dispenser and suggestions are made to formulate an instruction protocol that would help overcome these problems. The problems are, among others, 1°. The drops are not dispensed accurately into the eye; 2°. The distance between the eye and eye-drop dispenser is too great for fear of touching the eye, which makes applying the drops more difficult and increases the chance of inadvertent blinking; 3°. It is increasingly difficult to squeeze the eye-drop dispenser when the dispenser is nearly empty, consequently the hands start shaking and drops are not accurately inserted and/or more than one drop is inserted; 4°. The administration of eye-drops is even more difficult on account of shaking hands or arthritis which is common in elderly patients; it is difficult for arthritic patients to squeeze a dispenser, tilt their heads back and position the dispenser over the eye; 5°. Poor eyesight, common in the elderly makes it hard to administer eye-drops; 6°. A high probability that the eyedropper comes into contact with the eye, the eyelids, or the eyelashes; 7°. The possibility that hands have not been washed prior to the application of the eyedrops.

Notwithstanding the fact that there are numerous aids available on the market designed to help patients to self-administer eye-drops or sprays, the problems inherent in this method of administrating fluid medication have not been solved. There is still a need for an efficient aid for the self-administration of eye-drops and the like, which can be used universally with almost any kind of packing and which avoids or solves the problems as discussed in the literature as much as is possible. The present invention constitutes such an aid.

Surprisingly, it has now been found that problems occurring during the self-administration of eye-drops could be largely or entirely avoided by making one or more improvements to the housings of ophthalmic devices belonging to the state of the art, thereby facilitating considerably the self-administration of a fluid medication including an eye-spray.

The invention is characterised in that the housing further comprises an elongated support for the dispenser extending outwardly from the base portion into a direction remote from the eye, that the base portion further comprises a second opening providing a target for directing the eye upon administering the fluid eye medication to the eye, and that said base portion has, on its side directed towards the eye, an entirely or partially reflecting and/or colour-discriminating surface.

Preferably, the dispenser is engaged to the support by hand permitting both holding the device and dispenser between thumb and one or more fingers and squeezing the dispenser for release of its content, usually in the form of one or more droplets.

In another aspect of the invention at least one positioning member is positioned below the eye of the user in its close vicinity. In another and preferred aspect of the present invention a second positioning member is provided which is positioned above the eye on or in close vicinity of the eyebrow of the user.

In one embodiment of the invention the side of the base portion which is directed towards the eye is preferably entirely or partially provided with reflecting material, so that the user can see his/her eye for proper orientation. In actual practice it is convenient when the nozzle of the dispenser just covers the reflection of the eye of the user or rather his pupil which is an indication that the dispenser can be squeezed to release the fluid to the eye in a proper way.

In a preferred embodiment of the invention the reflecting surface is curved such that the eye is reflected and magnified. The reflecting surface may extend circularly in whole or in part. This surface enables a patient who self-administers eye-drops or eye-spray to aim more accurately thus being able to administering eye-drops more efficiently, and entirely, or almost entirely avoiding waste.

According to another preferred embodiment of the invention the base portion concerned is entirely or partially manufactured in colour and preferably in a monochromatic colour. In principle this colour may be any desired colour although it appears to be most appropriate when the base portion is made in red. If desired, characters or indications (for instance arrows) may also be assembled on a reflecting or non-reflecting surface.

Especially people with glaucoma experience difficulties with the self-administration of eye-drops since they are mostly older, have poor eyesight, and may have poor fine motor control. A trial revealed that this group derives great benefit from a device designed according to the invention described here, especially when produced with a red base portion.

The base portion may be assembled flat as well as somewhat curved (for instance circularly concave or convex or cylindrical-concave), particularly in order to perceive a normal, respectively magnified or reduced reflection. When the part to the side of the eye is entirely or partially reflecting, it is preferably assembled so as to perceive a magnified reflection of the eye. This can be achieved by making the surface somewhat concave (for instance circularly concave or cylindrical-concave).

In a particular preferred embodiment of the invention the ophthalmic device as a whole has a single-colour appearance which is preferably the colour of the colour-discriminating surface of the base portion. Evidently, this will facilitate and economize the production of the device.

The angle between the base portion of the device and the dispenser of the fluid medication may vary. Although an angle of approximately 90° is preferred in principle where assembly methods are concerned, it means that the patient has to be able to tilt his head far backwards in order to administer the liquid to the eye accurately. Furthermore self-administration means that the patient has to lift the arm high in order to position the hand above the head. In practice it appears that this causes problems for many patients.

According to a preferred embodiment of the invention the angle between the base portion and the centre line of the dispenser will be approximately between 30°-80°, with more preference for approximately 45°-75°, and most preference between approximately 60°-70°. This allows the above-mentioned problems to be entirely, or for the most part, solved. In this version it is efficient to adjust the base portion slightly, so that the device can be appropriately attached to the dispenser. Various aspects of the present invention will be explained further hereafter with reference to the described drawings.

The preferred angle between the base portion and the positioning member is approximately 9020 , but it will be apparent to the persons skilled in the art that numerous changes may be made in such details without departing from the spirit and scope of the invention.

In accordance with one embodiment of the invention the device is attached to the fluid medication dispenser through the opening in the base portion, and is essentially tightly fitted with that. For this purpose a suitable dispenser, for example an eye-drop-dispenser, has for instance a slightly tapered spout or nozzle, ending in a discharge orifice, which nozzle is put through the opening of the base portion of the device whereupon the device is assembled by means of the conical construction of the nozzle or is otherwise attached to the nozzle of the dispenser, preferably clipped and sealed.

It will be apparent to experts in the field that there are various possibilities for attaching the device to the dispenser in accordance with the invention. Naturally, the assembly also depends on the shape of the dispenser. Besides the above-mentioned assemblage it is possible to allow for variations in the size of the opening of the base portion so that, for instance, the dispenser can be screwed onto the opening. Also the opening may be assembled with a ring made of elastic material such as rubber in order to seal and/or strengthen the opening. The opening of the base portion is preferably situated in the centre of the part, however it may also be situated in an alternative position if desired.

It is further noted that when the ophthalmic device is provided with a support, as described above, which is a preferred embodiment of the invention, it is not necessary to attach or secure the dispenser or rather its nozzle to the first opening of the base portion. The engagement of the dispenser to the support between the thumb and one or more fingers of the user appears to be sufficient and also very effective since this position of the hand and the finger is also the right position for squeezing the dispenser to release the desired amount of its fluid.

According to another embodiment and in accordance with the invention the device may form a part of the packing. The design of the device remains essentially in accordance with the invention but it will be apparent to those of skill in the art that some modifications are deemed necessary in order to assemble the device as part of the packing. This will be further explained hereafter with reference to the described drawings. One of the possible modifications provides the opening in the base portion with a narrow flow opening so that the fluid may be better dosed. Furthermore, the device that is attached to the dispenser may be provided with a locking device in order to close the supply of fluid or spray intended for the eye. Such locking device usually consists of a screw cap or a cap with a click fastening.

In accordance with the invention the device can be applied in different ways. According to one embodiment a first positioning member is positioned onto the cheekbone under the eye and a second, optional member is positioned above the eye on or near the eyebrow. According to another embodiment a first positioning member is placed on the lower eyelid and in such a manner that the eyelid will be retracted somewhat. The advantage of this method is that the fluid drops from the dispenser into the lower conjunctival sac and there is little or no waste of medication.

The second opening in the base portion provides a target for directing the eye upon administering the fluid eye medication towards the eye, for example the ceiling or a light source. It is advantageous that the device is not closed around the eye since the separate positioning member(s) permits sufficient light to enter for the user to reflect the light from the base part.In accordance with the invention the device may be fabricated from a range of materials. Preferably a soft synthetic material is predominantly utilised, such as low-pressure polythene. Other synthetic materials, which may be mainly soft or stiff, are also appropriate such as polypropylene for example, ABS, PVC, polystyrene, or (co) polymers of this type. (Meth)acrylate may be utilised to obtain a transparent fabrication. The thickness of the material may well lie within certain limits, preferably within 0.5 to 3 mm, more particularly 1.5 to 2.5 mm. Preferably the rim of the positioning member that is placed on the side of the body, will be rounded off and thickened somewhat so as to prevent any physical discomfort.

From the foregoing discussion, it will be apparent to those skilled in the art that the device, according to the design of the invention can be applied efficiently and universally and therefore effects improvements to the current state of technology.

The description of the invention, which follows, together with the accompanying drawings, should not be construed as limiting the invention to the example shown and described.

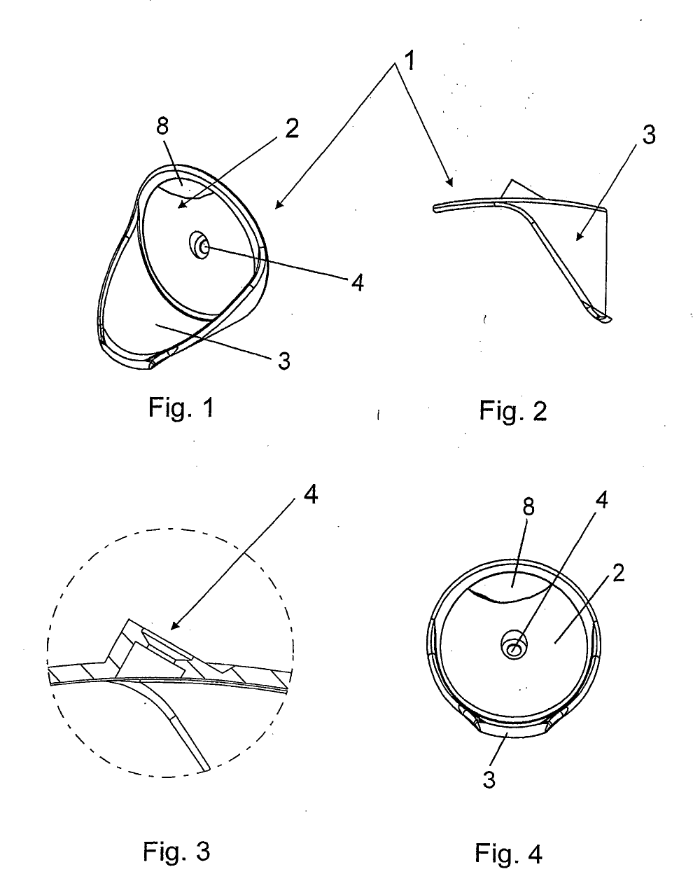

FIG. 1 is a perspective view of a first embodiment according to the invention.

FIG. 2 shows a side-view of the device of FIG. 1.

FIG. 3 is a schematic partial detail view of FIG. 2, wherein the assembly of the device on the fluid dispenser is rendered under a non-perpendicular angle.

FIG. 4 is a front elevational view of the device of FIG. 1 from the side of the positioning member.

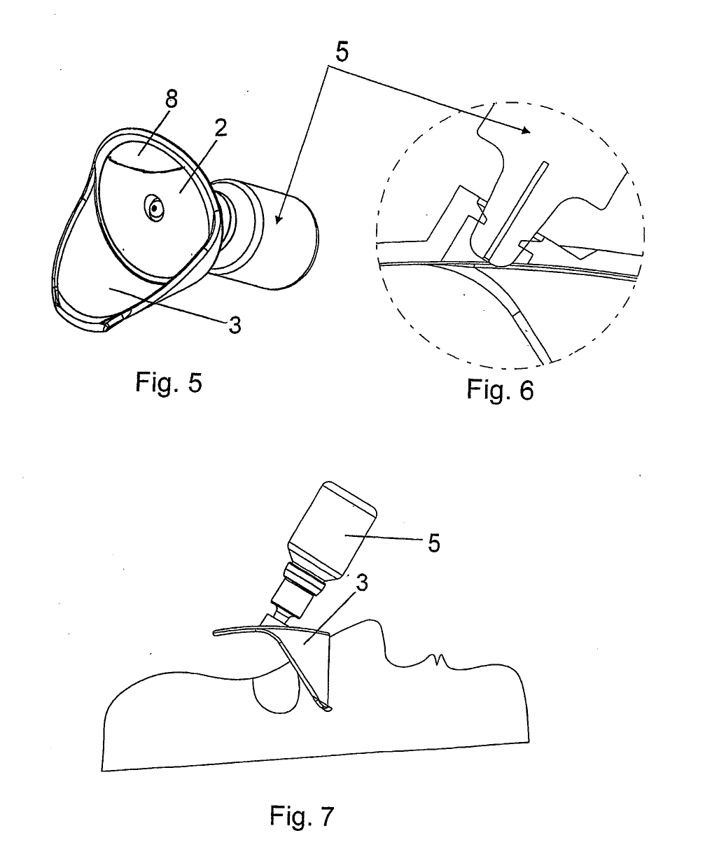

FIG. 5 shows the device of FIG. 1 in combination with a dispenser for fluid medication in the shape of an eye-drop dispenser.

FIG. 6 shows a schematic partial detail view of the assemblage of the device, according to the invention, upon the eye-drop dispenser of FIG. 5.

FIG. 7 is a sketch rendering of the combination of FIG. 5 of the application position on the patient's head.

FIG. 8 shows an alternative embodiment of the device according to the invention as part of the packing, complete with fluid dispenser and closing-cap.

FIG. 9 shows a schematic partial detail view of FIG. 8 with the fitted opening in the base portion for the fluid dosage.

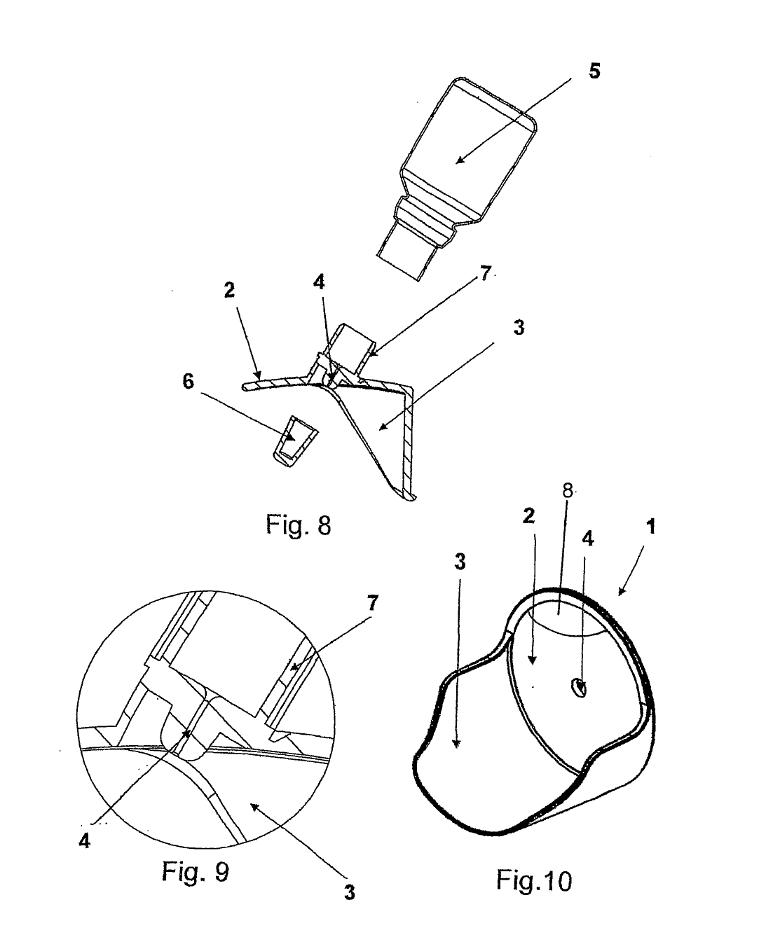

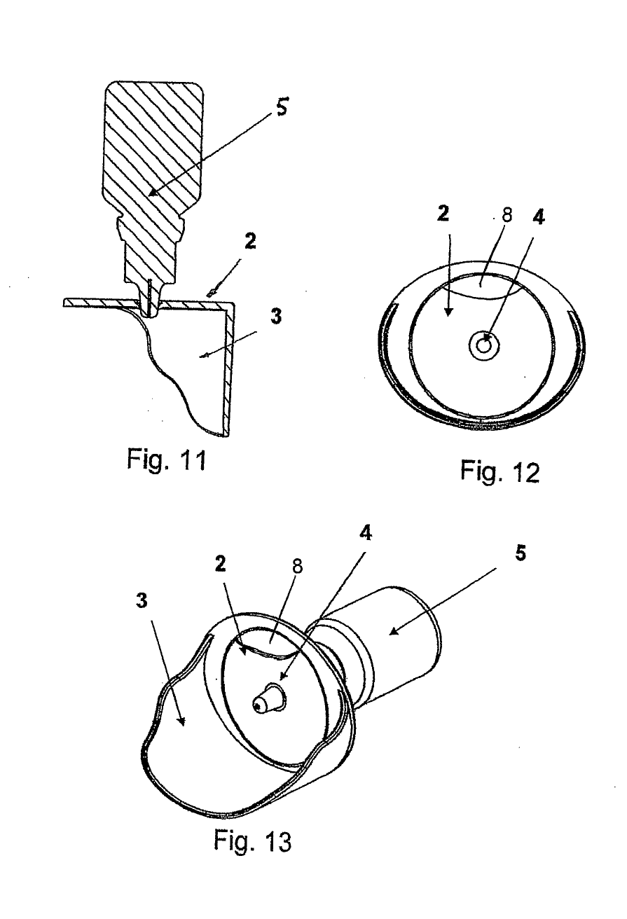

FIG. 10 is a perspective view of an alternative embodiment of the device according to the invention wherein a fluid dispenser is essentially assembled perpendicularly on the circular base portion, which is further illustrated schematically in FIG. 11.

FIGS. and 12 and 13 show a variant of the embodiment of FIG. 10, wherein the base portion is assembled oval-shaped.

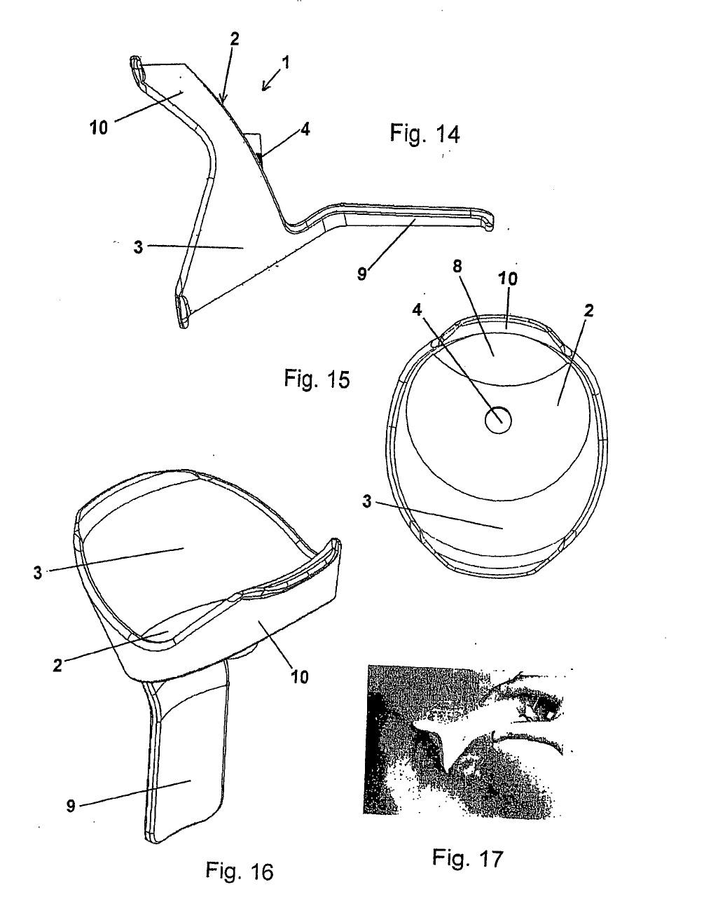

FIG. 14 is a side view of a preferred ophthalmic device according to the invention having both an upper and lower positioning member, and a support for the dispenser.

FIG. 15 is a front view of the preferred ophthalmic device of FIG. 14.

FIG. 16 is a perspective view of the device of FIG. 14; and

FIG. 17 is a photograph demonstrating the use of the preferred device shown in FIGS. 14-16, together with a dispenser for fluid eye medication.

In the various figures the same reference characters indicate the same or similar parts.

FIGS. 1, 2 and 4 show a first embodiment of an ophthalmic device of the present invention. The device comprises a housing 1, a base portion 2, which is depicted circularly in this figure. The base portion 2 in this embodiment is curved cylindrically whereby the centre line of the cylinder preferably runs horizontally, that is to say, essentially parallel to the tangent plane of the positioning member 3, which is discussed hereafter. The base portion 2 is preferably on the inner side around the opening 4 in whole or in part, provided with a reflecting surface, whereby when in use the eye is reflected and magnified and the fluid from the dispenser 5 (see for instance FIGS. 5 and 8) can be applied more accurately onto the eyeball.

On the contour of the base portion 2 a positioning member 3 is assembled, whereby one outer end stretches from the base portion and the other outer end is placed against the head in the proximity of the eye. The positioning member 3 has in principle the same shape in axial direction as the outer contour of the base portion 2. This shape is preferably circular or oval, whereby a good universal fit of the device near the eye of the user is obtained. The positioning member 3 is to the side of the eye, furthermore, is provided preferably with rounded angles and possibly a thickening, and is finished in such a manner that it can be held fast against the head without irritation. It is efficient to place the positioning member 3 to the bottom of the eye and preferably on the lower eyelid, although other locations (see for instance FIG. 7) are not excluded. By placing the positioning member 3 on the lower eyelid, the eye is held open forcibly, facilitating the application of the fluid efficiently and virtually without any waste. The base portion 2 further comprises an opening 4, where the opening of the dispenser 5 for fluid medication is put through. Preferably the device with the housing 1 according to the invention is attached 4 with a nozzle or spout of the dispenser 5 through this opening. The base portion 2 further has a second opening 8 which enables the user of the device to conveniently direct the eye to a target, e.g. a ceiling or light source, during application of material from the dispenser 5 to the eye. This second opening 8 is particularly useful when the device is provided with a second positioning member, as is shown below in the preferred embodiment of FIGS. 14-17.

FIGS. 2, 3, and 6 show details of the preferred embodiment, wherein the device with the housing 1 is assembled under a slanting angle (with regard to the base portion 2) on the dispenser 5. FIG. 3 shows particularly that the base portion 2 of device 1 includes an additional construction provision, whereby the device is attached firmly to the dispenser under the angle concerned via a nozzle (or spout) of the dispenser 5. The opening 4 of the base portion 2 is produced preferably with a flexible rim or an additional flexible ring, in order to provide appropriate closure and/or support.

FIGS. 8 and 9 show an embodiment wherein the device according to the invention is part of the packing and forms a whole—here: detachable—with the dispenser 5. Although the principle of this device is the same as the above-mentioned device this embodiment preferably includes some modifications, particularly with regard to the assemblage of the device on the dispenser, whereby the base portion 2 is provided with an additional assemblage element 7, which allows for more firmness due to the (semi-) permanent nature of the combination device/dispenser. The nozzle of the dispenser 5 is in this embodiment replaced by a narrowed opening 4, which functions as such. Further, the combination and the dispenser are sealed with a closure assembly 6, which usually has the shape of a screw or a clickcap.

FIGS. 10-13 show some alternative embodiments of the ophthalmic device with the housing 1 whereby the base portion 2 is fabricated flatly and the housing 1 is essentially assembled perpendicularly on the (centre line of the) dispenser 5. Therefore the construction of the base portion 2 can be assembled more simply, however this version has the disadvantage in that, in practice, the patient has to tilt his head far back in order to administer the fluid appropriately from the dispenser.

FIGS. 14-17 show a preferred embodiment of the device of the invention. As compared to the previous embodiments the extra features are the support 9 and a second positioning member 10 which further facilitate the use of the device.

It is to be understood that the present invention is not limited to the versions described and showed herein. Further modifications of the invention disclosed will occur to those skilled in the art and all such modifications and equivalents are deemed to be within the scope of the invention as defined by the following claims.

1. Ophthalmic device for administering fluid eye medication from a dispenser (5) to a human eye, said device comprising a housing (1) comprising a base portion (2) having a first opening (4), whereby the dispenser (5) is releasably engagable with the base portion (2) and the nozzle of the dispenser is assemblable through the said first opening (4), and at least one positioning member (3) extending outwardly from the base portion (5) having an edge at the outer end to be placed against the head of a user adjacent to the eye to keep the said dispenser (5) at a predetermined distance from the eye, characterised in that the housing (1) further comprises an elongated support (9) for the dispenser (5) extending outwardly from the base portion (2) into a direction remote from the eye, that said base portion (2) further comprises a second opening (8) providing a target for directing the eye upon administering the fluid eye medication to the eye, and that said base portion (2) has, on its side directed towards the eye, an entirely or partially reflecting and/or colour-discriminating surface.

2. Ophthalmic device according to claim 1, wherein said at least one positioning member (3) is positioned below the eye of the user in its close vicinity.

3. Ophthalmic device according to claim 2, further comprising a second positioning member (10) which is positioned above the eye on or in close vicinity of the eyebrow of the user.

4. Ophthalmic device according to claim 1, wherein the device has a single-colour appearance which is the colour of the colour-discriminating surface of the base portion (2).

5. Ophthalmic device according to claim 1, wherein the dispenser (5) is engaged to the support (9) by hand.

6. Ophthalmic device according to claim 1, wherein the said second opening (8) providing a target for directing the eye upon administering the fluid eye medication to the eye has a substantially larger diameter than the opening (4) for receiving the nozzle of a dispenser.

7. Ophthalmic device according to claim 1, wherein the base portion (2) is essentially provided with a reflecting surface which is curved such that the eye is reflected and magnified.

8. Ophthalmic device according to claim 1, wherein the base portion (2) is essentially circular or essentially oval-shaped.

9. Ophthalmic device according to claim 1, wherein the angle between the base portion (2) and the centre line of the dispenser (5) is between about 30° and about 90°, preferably between about 45° and about 80°, and most preferably between about 60° and 75°.

10. Ophthalmic device according to claim 1, wherein the dispenser (5) is part of the device.

11. Ophthalmic device according to claim 10, wherein the opening (4) is narrowed.

12. Ophthalmic device according to claim 10, which is provided with a closure assembly for the dispenser.
